(19) United States
(12) Patent Application Publication
(10) Pub. No.: US 2010/0104615 A1
(43) Pub. Date: Apr. 29, 2010

(54) ABSORBENT ARTICLE COMPRISING LACTIC ACID PRODUCING BACTERIA

(75) Inventor: Anna Weiner Jiffer, Danderyd (SE)

Correspondence Address: BIRCH STEWART KOLASCH & BIRCH, PO BOX 747, FALLS CHURCH, VA, 22040-0747, US

(73) Assignee: Ellen AB, Danderyd (SE)

(21) Appl. No.: 12/519,093

(22) PCT Filed: Dec. 14, 2007
(86) PCT No.: PCT/EP07/63925

§ 371 Date: Jul. 29, 2009

(30) Foreign Application Priority Data
Dec. 15, 2006 (SE) 0602712-2

(51) Int. Cl. A61K 35/74; A61L 15/36; A61P 31/04

(52) U.S. Cl. 424/402; 424/93.4; 424/93.45

(57) ABSTRACT

The present invention relates to absorbent articles comprising at least a liquid acquisition layer and an underlying liquid storage layer, said absorbent article comprising lactic acid producing bacteria. The lactic acid producing bacteria are dispersed in a hydrophobic carrier and the article further comprises at least one super-absorbent polymer. At least a part of the total amount of super-absorbent polymer and at least a part of the total amount of the bacteria are present in the liquid storage layer.

### FIELD OF THE INVENTION

The present invention relates to absorbent articles comprising lactic acid producing bacteria dispersed in a hydrophobic carrier, further comprising at least one super absorbent polymer, said absorbent article having a multi-layered structure comprising at least a liquid acquisition layer and an underlying liquid storage layer. The invention also relates to the use of such absorbent articles.

### BACKGROUND OF THE INVENTION

The skin of the urogenital tract and the urogenital mucus membranes of a healthy woman host a specific flora of beneficial and/or commensal microorganisms, such as various species of Lactobacillus. However, the urogenital tract can also be colonised by disease-causing microorganisms. The colonisation of unwanted microorganisms can be a result of sexual transmission, it can occur spontaneously or it can be the result of a disturbed normal microbial flora. The latter is, for instance, known to happen after certain antibiotic therapies.

Thus, the microbial flora of the female urogenital tract, such as in the vagina, may be disturbed and altered by a microbial infection, such as yeast (Candida albinancs), Trichomonas vaginalis, Neisseria gonorrhoeae, and Chlamydia trachomatis, and bacterial vaginosis (characterized by increased prevalence of Gardnerella vaginalis and Mobiluncus), an antibiotic treatment or other often complex causes.

During menstruation and sexual intercourse, the pH in the vagina is increased by the addition of blood and sperm, respectively. These fluids contain a lot of proteins, which may be digested by bacteria (e g Gardnerella vagnalis and Mobiluncus), which might establish in the vagina under conditions of increased pH. Degradation products, such as amines (e.g. putrescine and cadacerine) are then produced. At increased pH, these amines become volatile and present a “fishy” odour. Additionally, these women often have complaints of increased vaginal discharge and irritation. This condition is called bacterial vaginosis (BV), and is the most common condition associated with irritation and increased amount of odorous vaginal discharge (see Morris, M; Nicoll, A; Simms, I; Wilson, J; Catchpole, M, Bacterial vaginosis: A public health review, British Journal of Obstetrics and Gynaecology, 108(5):439-450, May 2001).

Bacterial vaginosis is the most common type of Vaginitis. Vaginitis is believed to be the result of displaced vaginal lactic acid producing bacteria which are replaced by a range of unwanted species such as Gardnerella vaginalis, Bacterioides, Mobiluncus, Prevotella bivia, and Mycoplasma hominis.

It is known that lactic acid producing bacteria of the Lactobacillus strain dominate the flora of healthy women, and that most of these Lactobacillus bacteria have an ability to sustain the growth and reduce the pathogenicity of many uropathogens.

It is also known that the antagonistic properties of Lactobacillus and other lactic acid producing bacteria against pathogens are at least partially denoted by their ability of producing different so called antimetabolites, such as lactic acid, hydrogen peroxide, bacteriocins, etc.

Prior art describe formulations, such as suspensions, suppositories and gelatine capsules, comprising viable lactic acid producing bacteria. Such formulations are for instance disclosed in U.S. Pat. No. 5,466,463 and WO 9 309 793. Furthermore, it is known to impregnate absorbent articles, such as tampons and sanitary napkins, with lactic acid producing bacteria for the purpose of preserving a normal flora of microorganisms in the urogenital tract of women, and thereby preventing urogenital infections, or regenerating a normal flora of microorganisms in the urogenital tract of women. Such a product is disclosed in EP 0 594 628.

However, lactic acid producing bacteria are sensitive to, for instance, moisture, temperature and oxidation.

WO 2002028446 discloses a process for manufacturing of a sanitary article comprising lactic acid producing bacteria that are viable under a long period of time. It is stated that a long shelf life is provided by means of one or more of the following factors:
(i) application of bacteria by gentle feeding, preferably extrusion,
(ii) dispersion of the bacteria in an essentially hydrophobic carrier,
(iii) application on and/or in a component that are to form part of the final article, and
(iv) application of a string of said dispersion of bacteria in and/or on the final sanitary article.

The essentially hydrophobic carrier, such as a fat, an oil, or a wax, reduces the mechanical stress on the bacteria, and acts as an adhesive between the bacteria and the component that the bacteria are applied to, and reduces contact between bacteria and air, moisture and water, i.e. it protects the bacteria from the environment.

It is preferred to apply the dispersion of bacteria it such a way that a major part of the bacteria is kept inside the final absorbent article. The bacteria are thereby better protected against the environment, for instance, against moisture and air than if applied onto the surface of the article.

An absorbent article, such as a diaper or panty liner or sanitary napkin or pad, usually comprises several different layers having different properties with respect to each other. Frequently, the absorbent structure at least comprises a liquid acquisition layer and a liquid storage layer. The liquid storage layer may comprise a cellulosic fluff pulp layer mixed with a super absorbent material, which are polymers having the ability to absorb water or bodily fluids many times their own weight. The liquid acquisition layer often comprises a porous fibrous layer of synthetic fibres.

In order to obtain a thin product, it is known to use a relatively high percentage of a super absorbent material in the absorbent structure. For example, it is known from EP 0 443 627 to use an absorbent structure containing 60 percent by weight of a super absorbent material.

As mentioned above, super absorbent materials are often used in absorbent articles due to their ability to absorb water or bodily fluids. However, e.g. WO 2005/032443, US 2005/0075617 and WO 00/59556 disclose the use of super absorbent materials for the purpose of inhibiting the growth of bacteria; i.e. the super absorbents may exhibit an antimicrobial effect.

As described above, in order to obtain absorbent articles comprising lactic acid producing bacteria with good viability, it is advantageous to disperse the bacteria in a hydrophobic carrier. A disadvantage is, however, that the hydrophobic carrier might have a negative impact on the rate of diffusion and release of the bacteria to the hydrophilic environment.

As also described above, it is suitable to arrange the bacteria within the interior of the absorbent article in order to provide a good bacteria viability. Moreover, for esthetical reasons the red-brownish bacteria are preferably not visible for the user. However, this arrangement results in that the bacteria have to migrate a long distance to reach the user, which also has a negative impact on the diffusion and release rate of the bacteria.

Thus, it would be a great advantage if the diffusion and release rate of lactic acid producing bacteria dispersed in a hydrophobic carrier, and optionally arranged within the interior of an absorbent article, could be increased within reasonable time frame, i.e. normal usage, 2-8 hours.

### SUMMARY OF THE INVENTION

The object of the present invention is to overcome at least part of the above mentioned problem. This object is achieved by an absorbent article comprising lactic acid producing bacteria dispersed in a hydrophobic carrier, further comprising at least one super absorbent polymer. The absorbent article has a multi-layered structure comprising at least a liquid acquisition layer and an underlying liquid storage layer. At least a part of the total amount of said super-absorbent polymer and at least a part of the total amount of said bacteria are present in the liquid storage layer.

The present inventors have found that the use of a super-absorbent polymer in combination with lactic acid producing bacteria dispersed in a hydrophobic carrier increases the rate of diffusion and release of the bacteria from the absorbent article. It is surprising and positive that application further away from the liner surface, i.e. in the liquid storage layer has an improving effect on the bacteria's ability to migrate to the surface, even after short time of usages. The superabsorbent polymer provides an acidic and dry environment for the bacteria and the bacteria are then activated upon contact with the fluid. Without being bound by any theory, it is believed that since the super-absorbent polymer is hydrophilic, it will help the bacteria to reach the surface of the absorbent article.

In a preferred embodiment of the present invention, the bacteria are dispersed in a hydrophobic carrier and applied together, or separate, with a super-absorbent polymer in an intermediate layer, e.g. the liquid storage layer of the absorbent article, preferably arranged under a top layer and one or more other layers, thereby protecting the bacteria from the surrounding environment, e.g. moisture, and increasing their survival. During production of the absorbent article, the bacteria are thus applied in the last production step, which results in less stress on the bacteria. Furthermore it allows for an easy and inexpensive manufacturing method. Another advantage is that the red brownish bacteria are not visible for the user of the absorbent article.

The present inventors have surprisingly found that an advantage of arranging the lactic acid producing bacteria together with a superabsorbent polymer, e.g. in the liquid storage layer is that the bacterial survival is enhanced. This is noteworthy and surprising since several prior art documents state that the beneficial effect of super absorbent materials is that these materials display an anti-microbial effect.

The invention also relates to the use of a super-absorbent polymer in an absorbent article for increasing the diffusion and release rate of lactic acid producing bacteria dispersed in a hydrophobic carrier, and optionally present within the interior of the absorbent article. Such lactic acid producing bacteria are applied together with a super-absorbent polymer.

### BRIEF DESCRIPTION OF THE DRAWINGS

FIG. 1 is a schematic view illustrating an absorbent article according to a preferred embodiment of the present invention.

FIG. 2 is a cross sectional view of the absorbent article in FIG. 1 across the line II.

### DETAILED DESCRIPTION OF THE INVENTION

As used herein the term “lactic acid producing bacteria” means bacteria that by fermentation produce lactic acid, preferably from the genera Lactobacillus, Leunoconostoc and Pediococcus, and any combination thereof. Examples of suitable strains, hereinafter referred to as LN-bacteria, are disclosed in WO2003038068. Such bacterial strains comprise the strain of Lactobacillus gasseri, denoted by the applicant as LN 40, deposited under number LMG P-20560, the strain of Lactobacillus casei subsp rhamnosus, denoted by the applicant as LN 113, deposited under number LMG P-20562, the strain of Lactobacillus fermentum, denoted by the applicant as LN 99, deposited under number LMG P-20561 the strain of Lactobacillus crispatus, denoted by the applicant as LN 01, deposited under number LMG P-20558, and the strain of Pediococcus acidilactici, denoted by the applicant as LN 23, deposited under number LMG P-20559 or variants thereof having essentially corresponding phenotypic and/or genotypic characteristics.

The bacteria may further advantageously be freeze-dried, in order to increase the survival of the bacteria.

In particular, the bacteria may originate from the urogenital tract of a woman with a normal flora of microorganisms.

As used herein the term “absorbent article” means tampons (both digital tampons and tampons with an applicator), sanitary napkins, panty liners, diapers, incontinence pads and the like.

As used herein the term “carrier” means a substance in which the bacteria may be dispersed. Such a substance is preferably semi-solid/solid at ambient conditions. However, it might also be an aqueous or non-aqueous liquid or solution.

The carrier is hydrophobic. As used herein the term “hydrophobic” means water-repellent.

Most preferably the carrier comprises fatty acids, i.e. the carrier may be a fat, an oil, a wax, etc.

First of all, a carrier comprising fatty acids is hydrophobic.

Secondly, a carrier comprising fatty acids may exist both in molten, semi-solid or solid form. It is preferred that the carrier is a fat in semi-solid/solid form at normal conditions, i.e. at ambient temperature, since the absorbent article then is easier to handle for e.g. the user. Further, to obtain a homogeneous dispersion the bacteria are preferably added to a carrier that is either in the molten state or semi-solid. A semi-solid substance is also easier to handle, e.g. to pump, during the manufacturing of the aforementioned absorbing sanitary article.

To allow release of bacteria from the carrier during use of the absorbent article, the fat should be in melted form at use of the sanitary article. Thus, the fat should preferably have a melting temperature between approximately 25° C. and 45° C., more preferably 30-37° C. This melting temperature range is also preferred with consideration to preparation and application of the dispersion. As known to a skilled person in the art, the bacteria do not survive high temperature.

When the lactic acid producing bacteria are released in the urogenital tract they propagate, and the advantages of lactic acid producing bacteria according to the introduction are thus obtained.

As used herein the term “dispersed” means that at least two phases are mixed. One phase constitutes of essentially solid particles (the dispersion is a suspension) or liquid (the dispersion is an emulsion), and this phase is dispersed in the other phase (the continuous phase).

As used herein the term “super-absorbent polymer” means materials that form hydrogels on contact with water (e.g., with urine, blood, and the like). One preferred type of hydrogel-forming, absorbent gelling material is based on poly acids, especially polyacrylic acid. Hydrogel-forming polymeric materials of this type are those which, upon contact with fluids (i.e., liquids) such as water or body fluids, imbibe such fluids and thereby form hydrogels. These preferred absorbent gelling materials will generally comprise substantially water-insoluble, slightly cross-linked, partially neutralized, hydrogelforming polymer materials prepared from polymerizable, unsaturated, acid containing monomers.

Further examples of such super-absorbent polymers are hydrolysed starch-acrylonitrile graft copolymers, starch acrylic acid graft copolymers, saponified vinyl acetate-acrylic ester copolymers, hydrolysed acrylonitrile copolymers, hydrolyzed acrylamide copolymers, ethylene maleic anhydride copolymers, isobutylene-maleic anhydride copolymers, poly-(vinylsulfonic acid), poly(vinylphosphonic acid), poly(vinylphosphoric acid), poly(vinylsulfuric acid), sulfonated polystyrene, poly(aspartic acid), polylactic acid, and any combination thereof.

The super-absorbent polymers may be in any form, for example as beads, granules, a foam, fibers, threads, and/or a film.

The amount of super-absorbent polymer in the absorbent article may be in the range of 5 to 70% by weight of the total dry weight of the absorbent article. Typically, the amount of super-absorbent polymer is 10 to 70% by weight, e.g. 20 to 70% by weight of the total dry weight of the absorbent article.

When the amount of super-absorbent polymer is too low, e.g. less than 5% by weight, the rate of diffusion and release of bacteria from the absorbent article is reduced. Furthermore, since superabsorbents have shown to have a positive effect on the bacterial survival, an amount of less than 5% by weight might result in that the bacterial survival is impaired. In contrast, the amount of super-absorbent polymer preferably does not exceed 70% by weight, since too much super-absorbent makes the layer in which the super-absorbent resides too “thick” and more difficult to seal, and might cause discomfort for the user.

As used herein, the term “therapeutically effective amount” means an amount that will lead to the desired therapeutic effect.

In some embodiments of the invention, a desired therapeutic effect obtained when using the absorbent article according to the invention is prophylaxis and/or treatment of infections of the urogenital tract, such as bacterial vaginosis or any other bacterial disorder in the vagina.

Thus, the absorbent article according to the present invention may be used for prophylaxis and/or treatment of bacterial vaginosis or any other bacterial disorder in the vagina.

The absorbent article may further be used for prophylaxis and/or treatment of infections of the urogenital tract, to prevent, alleviate the effects of, and/or treat a microbial infection.

In yet another aspect, the absorbent article according to the present invention may be used for prophylaxis and/or treatment of vaginal candidiasis.

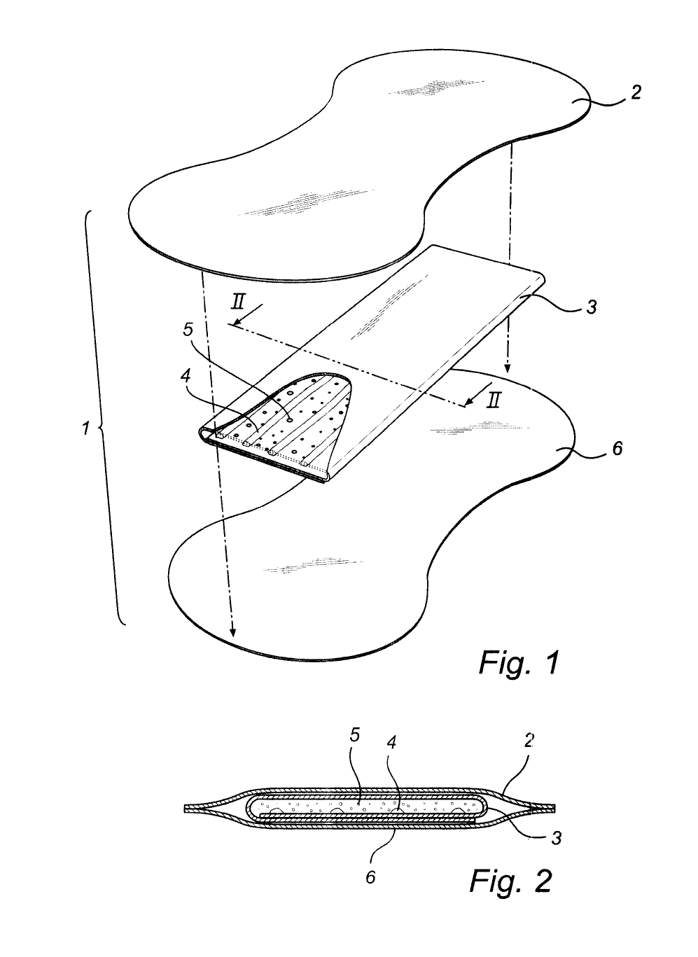

According to the invention, at least part of the bacteria are present within the interior, i.e. in the liquid storage layer of said article, and are thus protected from the environment. As is illustrated in FIG. 1, an absorbent article 1 according to a preferred embodiment of the present invention comprises more than one layer, i.e. at least a liquid acquisition layer 2 and a liquid storage layer 3 arranged beneath the liquid acquisition layer 2. The liquid storage layer 3 may, for instance, comprise a cellulosic fluff pulp layer mixed with a super absorbent material. Preferably, the liquid storage layer has a folded structure in which lactic acid producing bacteria dispersed in hydrophobic carrier are applied in strings 4 together with at least one superabsorbent polymer 5. As is illustrated in FIG. 1 and in FIG. 2, the strings of bacterial dispersion 4 and the superabsorbent polymer 5 are enclosed in a compartment formed from the liquid storage layer 3 within the absorbent article 1. Hence, the bacteria are better protected from moisture and air and the bacterial survival is further enhanced.

The bacteria dispersed in hydrophobic carrier are typically applied in 1-4 strings on the liquid storage layer 3. This is beneficial since the bacteria are surrounded by carrier material which protects them from air and moisture and also increases the migration rate of the bacteria to the surface of the absorbent article, i.e. the surface facing the user.

The liquid acquisition 2 layer may, for instance, comprise a porous fibrous layer of synthetic fibres.

Usually, the absorbent article 1 further comprises a backing sheet 6.

At least a part of the total amount of super-absorbent polymer and at least a part of the total amount of the bacteria are present in the liquid storage layer 3. Due to this arrangement, the bacteria will be protected from the environment, and at the same time the super-absorbent polymer will help in the diffusion of the bacteria to the surface of the absorbent article and release therefrom. Also, an increased bacterial survival is obtained by this arrangement, since the superabsorbent has shown to have a positive effect on bacterial survival.

In one embodiment of the present invention, more than 50% by weight of the total amount of super-absorbent polymer and more than 50% by weight of the bacteria are present in the liquid storage layer. This arrangement allows for an increased bacterial diffusion and survival, and for preventing red brownish stains on the surface of the absorbent article, i.e. the surface facing the user. Most preferably, essentially all bacteria are present in the liquid storage layer of the absorbent article.

The present invention also relates to the use of an absorbent article having the above mentioned features for use as a medicament.

Additionally, the invention relates to the use of said absorbent article in the manufacture of a medicament for the prophylaxis and/or treatment of:
bacterial vaginosis or any other bacterial disorder in the vagina.
infections of the urogenital tract.
vaginal candidiasis

The present invention also relates to an absorbent article for the prophylaxis and/or treatment of the above mentioned conditions.

### EXAMPLES

In vitro-studies were carried out to determine if LN-bacteria positioned in different layers and materials within the interior of a panty liner can migrate to the surface of the panty-liner over time.

For this test, commercially available panty liners were used. The bacteria mixture LN inside (see below) was applied within the interior of the panty liner. Three different panty liner were tested. Each type was prepared with LN-bacteria in different layers, depending on the construction of the liner. Every sample was prepared 24-72 hours before testing. The prepared samples were stored in ambient environment after preparation and before testing.

The samples were hydrated by PBS-water, 2-3 ml/samples. LN liners were subjected to an environment similar to normal usage for 2, 4, 8 and 24 hours; 35° C.-37° C. and controlled humidity of 55%.

After cultivation, the amount of bacteria and the number of different strains that had migrated to the surface of the panty liner after each time period was calculated.

### Test Material

### Liners:

### A Vivia Slipsuper Sensitive

B Viriana super active

### C Viriana Normal

TABLE 1

| | A Vivia | | |
|---|---|---|---|
| | Slipsuper | B Viriana super | |
| Name | Sensitive | active | C Viriana Normal |
| Weight | 2.30 g ± 5% | 2.30 g ± 5% | 2.30 g ± 5% |
| Compostion: | | | |
| Non woven | PE/PP | 3D-foil and PE | PP |
| | (layer 1) | | |
| Liquid acquisition | PE/PP | PE/PP | |
| layer | | | |
| | Layer 2 in | (layer 1 in | (layer 1) |
| | experiment | experiment) | |
| Liquid | Cellulose | Cellulose | Cellulose |
| storage layer | | (layer 2) | (viscose) |
| | | | bleached |
| | Layer 3 in | | (layer 2) |
| | experiment | | |
| Super-absorbent | — | Salt of | — |
| polymer | | cross fixing | |
| | | polyacrylic acid | |
| | | (layer 3) | |
| Liquid block | PE-foil | PE-foil | None |

Glue for attaching panty shield
Silicone paper strip, width 5.0 cm

### LN-Pool

The LN-bacteria consisted of grained powder of four different lyophilised bacterial strains, denoted by:

LN 23-10% (by weight)

### LN 40-36%

### LN 99-27%

### LN 113-27%

### LN Inside

LN-bacteria according to the above were mixed in hard fat. Approximately 3 kg LN-bacteria were mixed in 190 kg hard fat. The concentration of bacteria was 1E+9 cfu/g.

### Methods

### Sample Preparation

Each liner was prepared with 75 mg of LN inside/sample, i.e. 7.5·10⁷ CFU/sample. The LN inside was applied in different layers in different samples. In table 1, it can be seen that LN inside also could be applied between two layers.

### Testing

After storage, each liner sample was laid in a petri dish with ROGOSA agar. The samples were hydrated by 2-3 ml PBS buffer. Each sample was then loaded with a weight of 0.5-0.6 kg to simulate human usage.

The samples, including agar plates, were put in the incubator for 2, 4, 8 or 24 hours, respectively. After the test period each sample with ROGOSA agar plate was withdrawn from the incubator and the LN liner was removed from the agar-coated petri dish.

### Cultivation

The agar plates with diffused LN-bacteria were incubated in an anaerobic jar at 37° C. in 7-10% CO₂ for a minimum of 48 h, and a maximum of 72 hrs.

### Quantification of Colonies

Quantification of colonies was done by microscopy. All colonies per sample were calculated. In some cases the agar plates were so confluent that it proved to be impossible to count and/or distinguish different colonies from each other.

### Identification of Different Colonies

Colonies from each plate with highest non-confluent growth were selected and studied for genus determination of Lactobacillus (and Pediococcus). Genus was determined primarily by Gram staining, catalase reaction and microscopic examination in order to confirm Lactobacillus (or Pediococcus) morphology.

### Results

In the table below, the results of the testing and cultivation is presented. Each sample is presented in two cells; one for quantification of colonies and one illustrating the amount of different bacteria that had diffused to the surface after the test period.

The negative control was negative in all cases, i.e. the samples were not contaminated.

TABLE 2
Vivia Slipsuper sensitive

| | Layer | | | | | |
|---|---|---|---|---|---|---|
| | 1 | 1 | 2 | 2 | 3 | 3 |
| LN bact - 2-3 ml PBS added | colonies | different | colonies | different | colonies | different |
| 2 h | 2000 | .2-3 | 3000 | ≧3 | 1000 | ≧3 |
| 4 h | 3000 | ≧2 | 3000 | ≧3 | 2000 | ≧3 |
| 8 h | Confl | Confl | confl, 100 | ≧2 | confl, 100 | ≧2 |
| 24 h | Confl | .1-2 | confl, 100 | 1-2 | confl, 100 | .1-2 |

Super active

| | Layer | | | | | |
|---|---|---|---|---|---|---|
| | 1 | 1 | 2 | 2 | 3 | 3 |
| LN bact - 2-3 ml PBS added | colonies | different | colonies | different | colonies | different |
| 2 h | 4000 | ≧3 | 200 | 4 | 2000 | ≧3 |
| 4 h | 4000 | ≧3 | confl, 5000 | ≧2 | 4000 | ≧3 |
| 8 h | Confl | 1 | confl, >5000 | 1-2 | confl, >5000 | ≧3 |
| 24 h | Confl | 1 | confl, >5000 | ≧1 | confl, >5000 | .1-2 |

Viriana Normal

Layer

| | 1 | 1 | 2 | 2 |
|---|---|---|---|---|
| LN bact - 2-3 ml PBS added | colonies | different | colonies | different |
| 2 h | 700 | 3 | 60 | 3 |
| 4 h | 300 | 3 | 25 | 3 |
| 8 h | 500 | 3 | 7 | 2 |
| 24 h | 1000 | 3 | 7 | 3 |

Confl = confluent, different colonies that were not possible to distinguish from each other.

LN-bacteria diffused from each sample already after 2 hours. In most cases more than two different bacterial strain diffused to the surface at each time point. In many cases all strains were able to be identified on the surface.

### CONCLUSION

For manufacturing and esthetical reasons it is preferable to apply the bacteria as far down in the panty-liner as possible. This allows for an easy and inexpensive manufacturing method and for a panty-liner which does not display red brownish stains on the surface of the panty liner.

The super-absorbent polymer in the “super active” liner seems to have a positive effect on the diffusion and the survival of the LN-bacteria. It is surprising and positive to find that application further away from the liner surface, i.e. in the liquid storage layer, with a super-absorbent polymer, even seem to have an improving effect on the bacteria's ability to migrate to the surface, even after short time of usages. It is clear from table 2 that application of bacteria to the absorbent article containing a super-absorbent polymer results in a more rapid migration of bacteria to the surface of the absorbent article. This effect is clearly seen even when the bacteria are placed in layer 2 and 3 of the absorbent article. Hence, bacteria can be placed within the absorbent article and thus be protected from the environment, and at the same time rapid migration of the bacteria to the surface will be effected by use of a super-absorbent polymer.

1. An absorbent article comprising lactic acid producing bacteria dispersed in a hydrophobic carrier, further comprising at least one super absorbent polymer, said absorbent article having a multi-layered structure comprising at least a liquid acquisition layer and an underlying liquid storage layer, wherein at least a part of the total amount of said super-absorbent polymer and at least a part of the total amount of said bacteria are present in said liquid storage layer.

2. An absorbent article according to claim 1, wherein said absorbent article is selected from the group consisting of a sanitary napkin, a panty liner, a tampon, an incontinence protection and a diaper.

3. An absorbent article according to claim 1, wherein said super-absorbent polymer is selected from the group consisting of polyacrylic acid, hydrolysed starch-acrylonitrile graft copolymers, starch acrylic acid graft copolymers, saponified vinyl acetate-acrylic ester copolymers, hydrolysed acrylonitrile copolymers, hydrolyzed acrylamide copolymers, ethylene maleic anhydride copolymers, isobutylene-maleic anhydride copolymers, poly-(vinylsulfonic acid), poly(vinylphosphonic acid), poly(vinylphosphoric acid), poly(vinylsulfuric acid), sulfonated polystyrene, poly(aspartic acid), polylactic acid, and any combination thereof.

4. An absorbent article according to claim 3, wherein said superabsorbent polymer is polyacrylic acid.

5. An absorbent article according to claim 1, wherein said super-absorbent polymer is present in the form of beads, granules, a foam, fibers, threads, and/or a film.

6. An absorbent article according to claim 1, wherein the amount of said super-absorbent polymer is within the range of 10 to 70% by weight of the total dry weight of said absorbent article.

7. An absorbent article according to claim 1, wherein said hydrophobic carrier comprises fatty acids.

8. An absorbent article according to claim 1, wherein said bacteria are selected from the group consisting of Pediococcus, Lactobacillus, Leuconostoc, and any combination thereof.

9. An absorbent article according to claim 8, wherein said bacteria are selected from the group consisting of the strain of Lactobacillus gasseri, LN 40, deposited under number LMG P-20560, the strain of Lactobacillus casei subsp rhamnosus, LN 113, deposited under number LMG P-20562, the strain of Lactobacillus fermentum, LN 99, deposited under number LMG P-20561, the strain of Lactobacillus crispatus, LN 01, deposited under number LMG P-20558, and the strain of Pediococcus acidilactici, LN 23, deposited under number LMG P-20559, or variants thereof having essentially corresponding phenotypic and/or genotypic characteristics.

10. An absorbent article according to claim 1, wherein said bacteria originate from the urogenital tract of a woman with a normal flora of microorganisms.

11. An absorbent article according to claim 1, wherein said bacteria are freeze-dried.

12. An absorbent article according to claim 1, wherein more than 50% by weight of the total amount of super-absorbent polymer and more than 50% by weight of the bacteria are present in the liquid storage layer.

13. Use of a super-absorbent polymer in an absorbent article comprising lactic acid producing bacteria dispersed in a hydrophobic carrier, said absorbent article having a multi-layered structure comprising at least a liquid acquisition layer and an underlying liquid storage layer for increasing the diffusion and release rate of lactic acid producing bacteria dispersed in a hydrophobic carrier.

14. Use according to claim 13, wherein said bacteria are present in said liquid storage layer of said absorbent article and then migrate to the surface of said absorbent article.

15. An absorbent article according to claim 1 for use as a medicinal product.

16. Use of an absorbent article according to claim 1 in the manufacture of a medicament for the prophylaxis and/or treatment of bacterial vaginosis or any other bacterial disorder in the vagina.

17. Use of an absorbent article according to claim 1 in the manufacture of a medicament for the prophylaxis and/or treatment of infections of the urogenital tract.

18. An absorbent article according to claim 1 for the prophylaxis and/or treatment of bacterial vaginosis or any other bacterial disorder in the vagina.

19. An absorbent article according to claim 1 for the prophylaxis and/or treatment of infections of the urogenital tract.

20. An absorbent article according to claim 1 for use as a medical device.
